(19) United States
(12) Patent Application Publication
(10) Pub. No.: US 2010/0160439 A1
(43) Pub. Date: Jun. 24, 2010

(54) DERMATOLOGICAL COMPOSITIONS COMPRISING AT LEAST ONE RETINOID COMPOUND, AN ANTI-IRRITANT COMPOUND AND BENZOYL PEROXIDE

(75) Inventor: Claire MALLARD, Mougins (FR)

Correspondence Address: BUCHANAN, INGERSOLL & ROONEY PC, POST OFFICE BOX 1404, ALEXANDRIA, VA, 22313-1404, US

(73) Assignee: GALDERMA RESEARCH & DEVELOPMENT, BIOT (FR)

(21) Appl. No.: 12/635,866

(22) Filed: Dec. 11, 2009

Related U.S. Application Data
(63) Continuation of application No. PCT/EP08/05731, filed on Jun. 11, 2008.
(60) Provisional application No. 60/929,205, filed on Jun. 18, 2007.

(30) Foreign Application Priority Data
Jun. 11, 2007 (FR) 0755658

(51) Int. Cl. A61K 31/192; A61K 8/368; A61P 17/10; A61P 17/12; A61Q 19/00

(52) U.S. Cl. 514/569

(57) ABSTRACT

Dermatological compositions contain, formulated into a physiologically acceptable medium, at least one retinoid compound selected from among all-trans retinoic acid, isotretinoin, motretinide, and naphthoic acid compounds of formula (I), and salts and esters thereof:

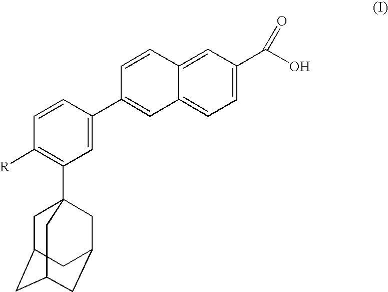

wherein R is a hydrogen atom, a hydroxyl radical, a branched or unbranched alkyl radical having from 1 to 4 carbon atoms, an alkoxy radical having from 1 to 10 carbon atoms, or a cycloaliphatic radical which is substituted or unsubstituted, and benzoyl peroxide, and also at least one anti-irritant compound selected from among 18β-glycyrrhetinic acid, and its salts and derivatives thereof.

### CROSS-REFERENCE TO PRIORITY/PROVISIONAL APPLICATIONS

This application claims priority under 35 U.S.C. §119 of FR 0755658, filed Jun. 11, 2007, §120 of U.S. Provisional Application No. 60/929,205, filed Jun. 18, 2007, and is a continuation/national phase of PCT/EP 2008/057310, filed Jun. 11, 2008 and designating the United States (published in the English language on Dec. 18, 2008 as WO 2008/152064 A1), each hereby expressly incorporated by reference in its entirety and each assigned to the assignee hereof.

### BACKGROUND OF THE INVENTION

2. Technical Field of the Invention

The present invention relates to compositions for topical application, and to the administration thereof as cosmetic or pharmaceutical products, said compositions being useful in the treatment of dermatological disorders, and in particular, in the treatment of acne.

2. Description of Background and/or Related and/or Prior Art

Acne is a common multi-factor pathology that attacks skin rich in sebaceous glands (face, shoulder area, arms and intertriginal areas). It is the most commonly occurring form of dermatosis. The following five pathogenic factors play a determining role in the formation of acne:

1. genetic predisposition;

2. overproduction of sebum (seborrhea);

3. androgens;

4. follicular keratinization disorders (comedogenesis); and

5. bacterial colonization and inflammatory factors.

There are several forms of acne, the common factor of all being attack of the pilosebaceous follicles. Exemplary are in particular, of acne conglobata, cheloid acne of the nape of the neck, acne medicamentosa, recurrent miliary acne, necrotic acne, neonatal acne, premenstrual acne, occupational acne, acne rosacea, senile acne, solar acne and common acne.

Common acne, also known as polymorphic juvenile acne, is the most common. It comprises four stages:

stage 1 corresponds to comedonic acne characterized by a large number of open and/or closed comedones and of microcysts;

stage 2, or papulopustular acne, is of mild to moderate seriousness. It is characterized by the presence of open and/or closed comedones, of microcysts, but also of red papules and pustules. It mainly affects the face and leaves few scars;

stage 3, or papulocomedonic acne, is more serious and extends to the back, the chest and the shoulders. It is accompanied by a large number of scars;

stage 4, or nodulocystic acne, is accompanied by numerous scars. It exhibits nodules and also painful voluminous crimson pustules.

The various forms of acne described above can be treated with active agents such as anti-seborrheic agents and anti-infectives, for example, benzoyl peroxide (in particular, the product Eclaran® marketed by Pierre Fabre), with retinoids such as tretinoin (in particular, the product Retacnyl® marketed by Galderma) or isotretinoin (the product Roaccutane® marketed by Laboratories Roche), or else with naphthoic acid derivatives. Naphthoic acid derivatives such as, in particular, 6-[3-(1-adamantyl)-4-methoxyphenyl]-2-naphthoic acid, which is commonly called adapalene (the product Differine® marketed by Galderma), are widely described and recognized as active ingredients that are just as effective as tretinoin for the treatment of acne.

However, to increase the effectiveness of treatments, especially treatments for dermatological disorders, and in particular, for acne, and to reduce the toxicity of the active ingredients (Cunliffe W. J., J. Dermatol. Treat., 2000, 1I (suppl2), S13-S14), several categories of active ingredients are commonly administered.

An article by Korkut and Piskin, J. Dermatology, 2005, 32: 169-173, reports the results of a study comparing a treatment combining application of adapalene in the evening and application of BPO in the morning, relative to an application of each of the active principles alone. The authors do not observe any superiority of the combined treatment over a period of 11 weeks of treatment.

Since the multiple application of various dermatological products is quite laborious and demanding for the patient, the value of a novel treatment which is effective on dermatological conditions, in particular, acne, in a stable composition which offers good cosmeticity, which significantly improves tolerance and which makes it possible to increase patient compliance, is therefore apparent.

Combinations of active agents are now beginning to appear. Exemplary is the DUAC combination comprising clindamycin and benzoyl peroxide or combinations of antibiotics. Among these, also exemplary is a gel comprising at least one retinoid and benzoyl peroxide as described in WO 03/55472.

However, the formulation of such a composition comprising several active agents, including benzoyl peroxide, presents several problems.

First, the effectiveness of the benzoyl peroxide is linked to its decomposition when it is brought into contact with the skin. It is the oxidizing properties of the free radicals produced during this decomposition which produces the desired effect. Thus, to maintain optimum effectiveness for the benzoyl peroxide, it is important to prevent its decomposition before use, i.e., during storage.

Now, benzoyl peroxide is a chemical compound that is unstable, which makes it difficult to formulate in finished products.

The solubility and the stability of benzoyl peroxide have been studied in ethanol, propylene glycol and various mixtures of polyethylene glycol 400 (PEG 400) and water (Chellquist E. M. and Gorman W. G., Pharm. Res., 1992, Vol. 9: 1341-1346). The authors thus note that benzoyl peroxide in solution degrades more or less rapidly in all the solvents studied, depending on the type of solvent and on its concentration.

The benzoyl peroxide degradation times observed are so short that they do not make it possible to formulate a product that is intended to be marketed.

It is known, moreover, that benzoyl peroxide is more stable in water and in propylene glycol when it is in suspension (i.e., in disperse form), since it is not degraded after 90 days of storage in these solvents.

Thus, to limit the problem of rapid instability of benzoyl peroxide in solution, it has been found to be advantageous to formulate benzoyl peroxide in dispersed form.

Another difficulty to be overcome in the formulation of a composition comprising both a retinoid, an anti-irritant and benzoyl peroxide is that most retinoids are particularly sensitive to natural oxidation, to visible light and to ultraviolet radiation. Since benzoyl peroxide is a strong oxidizing agent, the chemical compatibility of these compounds in the same formulation presents many problems of stability from the physical and chemical point of view.

In addition, it has been reported that benzoyl peroxide can sometimes induce dryness of the skin and on certain occasions irritation of the skin.

Among the retinoids commonly employed, adapalene in particular, exhibits a unanimously proven effectiveness. However, it would be advantageous and useful to reduce the irritation caused by retinoids applied topically, including adapalene, although its tolerance is greater than that of its competitors belonging to the same chemical category (tretinoin, tazarotene).

The term “irritation” means, in particular, the symptoms or the conditions linked to the application to the skin of chemical products of natural or synthetic origin, used in cosmetics or dermatology, and which can be characterized in particular, by an inflammation, an erythema, an oedema, redness, itching, pain, burning, a sting, or else tingling.

### SUMMARY OF THE INVENTION

The present invention solves the above problems by providing stable topical compositions that are barely irritant or not at all, comprising, in a pharmaceutically acceptable medium, a retinoid, benzoyl peroxide and an anti-irritant, useful for the treatment of dermatological disorders, and in particular, for the treatment of acne.

The presence of an anti-irritant makes it possible to significantly improve the tolerance of the subject compositions comprising a retinoid and benzoyl peroxide, and therefore to overcome the problem of irritation. Advantageously, such compositions according to the invention make it possible to increase the concentrations of the active ingredients while at the same time limiting their side effects. In addition, when said compositions are in the form of a gel, a cream-gel or an emulsion, same provide emollience and avoids in particular, leaving too greasy a feel on the skin.

In addition, the pharmaceutical or cosmetic compositions according to the invention conserve, throughout their shelf life, precise physicochemical criteria for guaranteeing their pharmaceutical or cosmetic quality. Among these criteria, it is necessary for the rheological properties to be conserved. These rheological properties define the behavior and the texture of the composition during application, but also the properties of release of the active ingredients.

Thus, novel compositions have now been developed which meet the above needs, while at the same time overcoming the problem of irritation.

The present invention thus features compositions comprising, formulated into a physiologically acceptable medium, at least one retinoid compound selected from among all-trans retinoic acid, isotretinoin, motretinide, and naphthoic acid compounds of formula (I), and salts and esters thereof:

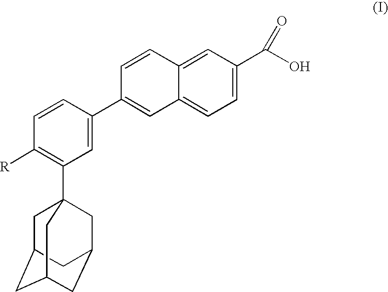

wherein R is a hydrogen atom, a hydroxyl radical, a branched or unbranched alkyl radical having from 1 to 4 carbon atoms, an alkoxy radical having from 1 to 10 carbon atoms or a cycloaliphatic radical which is substituted or unsubstituted, at least one anti-irritant compound selected from among 186-glycyrrhetinic acid (enoxolone), its salts and its derivatives, and benzoyl peroxide.

### DETAILED DESCRIPTION OF BEST MODE AND SPECIFIC/PREFERRED EMBODIMENTS OF THE INVENTION

In one particular embodiment of the invention, the retinoid compound is a naphthoic acid compound of formula (I), and salts and esters thereof.

In a specific embodiment, the naphtoic acid of formula (I) is such that the alkyl radical is the methyl, ethyl, propyl or butyl radical; the alkoxy radical is the methoxy, ethoxy, propoxy, butoxy, hexyloxy or decyloxy radical; and the cycloaliphatic radical is the 1-methylcyclohexyl radical or the 1-adamantyl radical.

In a preferred embodiment, the retinoid compound is selected from among 6-[3-(1-adamantyl)-4-methoxyphenyl]-2-naphthoic acid (adapalene), 6-[3-(1-adamantyl)-4-hydroxyphenyl]-2-naphthoic acid, 6-[3-(1-adamantyl)-4-decyloxyphenyl]-2-naphthoic acid and 6-[3-(1-adamantyl)-4-hexyloxyphenyl]-2-naphthoic acid and salts and esters thereof. More preferably, the retinoid compound is 6-[3-(1-adamantyl)-4-methoxyphenyl]-2-naphthoic acid (adapalene) and salts and esters thereof.

In a specific embodiment, the concentration of retinoid compound ranges from 0.001% to 10%, preferentially from 0.01% to 5%, preferably from 0.01% to 1%, more preferentially from 0.01% to 0.5%, and preferentially still from 0.1% to 0.3% by weight of the total weight of the composition. More preferred, the concentration of retinoid compound is equal to 0.1% or equal to 0.3%.

In a preferred embodiment of invention, the anti-irritant compound is selected from among the potassium salt of 18β-glycyrrhetinic acid, the sodium salt of 18β-glycyrrhetinic acid, the monoammonium salt of 18β-glycyrrhetinic acid, the disodium succinate salt of 18β-glycyrrhetinic acid, the dipotassium salt of 18β-glycyrrhetinic acid and the monoester of 18β-glycyrrhetinic acid.

Preferably, the concentration of anti-irritant compound ranges from 0.01% to 10%, preferentially from 0.1% to 7%.

In a specific embodiment of the invention, the benzoyl peroxide is in dispersed form in the composition. Alternatively, the benzoyl peroxide is in encapsulated or free form. Preferably, the composition comprises from 0.0001% to 20% of benzoyl peroxide, preferentially from 0.025% to 10%, even more preferentially from 2.5% to 5%.

The compositions are for topical application. Preferably, the composition is in the form of aqueous, aqueous-alcoholic or oily dispersions, dispersions of the lotion type, aqueous, anhydrous or lipophilic gels, emulsions of liquid or semi-liquid consistency of the milk type, obtained by dispersion of a fatty phase in an aqueous phase (O/W) or vice versa (W/O), or suspensions or emulsions of soft, semi-liquid or solid consistency of the cream, gel, cream-gel, foam or ointment type, or microemulsions, microcapsules, microparticles or vesicular dispersions of ionic and/or nonionic type, in the form of sprays, or else in the form of dermal devices such as patches.

More preferably, the composition is in the form of a gel, a cream-gel or an emulsion.

Most preferred, said composition is a medicament.

The present invention also features administration, whether regime or regimen of at least one retinoid compound, benzoyl peroxide and at least one anti-irritant compound selected from among 18β-glycyrrhetinic acid (enoxolone), its salts and its derivatives, or composition comprised thereof for the treatment and/or prevention of dermatological conditions linked to a keratinization disorder relating to cell differentiation and proliferation, in particular, for treating common acne, comedonic acne, papulopustular acne, papulocomedonic acne, nodulocystic acne, acne conglobata, cheloid acne of the nape of the neck, recurrent miliary acne, necrotic acne, neonatal acne, occupational acne, acne rosacea, senile acne, solar acne and acne medicamentosa. Preferably, the subject pharmaceutical compositions are useful for preventing, inhibiting or treating common acne.

The present invention also features a method for formulating a subject composition by mixing at least one retinoid compound with benzoyl peroxide and with at least one anti-irritant compound and in particular, in the form of a gel, and also a method for preparing a composition in the form of a cream-gel and/or a method for preparing a composition in the form of an emulsion.

This invention also provides a regime or regimen for treating and/or preventing and/or inhibiting dermatological conditions linked to a keratinization disorder relating to cell differentiation and proliferation, in particular, for treating common acne, comedonic acne, papulopustular acne, papulocomedonic acne, nodulocystic acne, acne conglobata, cheloid acne of the nape and/or the neck, recurrent miliary acne, necrotic acne, neonatal acne, occupational acne, acne rosacea, senile acne, solar acne and acne medicamentosa, comprising administering to an individual in need thereof, a therapeutical effective amount of composition defined previously.

Finally, the present invention features a non-therapeutic cosmetic treatment process for embellishing the skin or its surface appearance, in which a composition comprising, formulated into a physiologically acceptable medium, a retinoid, an anti-irritant selected from among 18β-glycyrrhetinic acid (enoxolone), its salts and its derivatives and benzoyl peroxide is topically applied to the skin and/or its integument annexes. In a preferred embodiment, the treatment of skin is for skin with an acneic tendency or for combating the greasy appearance of the skin or the hair.

Herein unless otherwise specified, it is understood that, when concentration ranges are given, they include the upper and lower limits of said range. Similarly, unless otherwise indicated, the proportions of the various constituents of the composition are expressed as percentage by weight (m/m) of the total weight of said composition.

According to the invention, the subject compositions comprise, in a physiologically acceptable medium, at least one compound of retinoid type, at least one anti-irritant compound and benzoyl peroxide.

The term “physiologically acceptable medium” means a medium compatible with the skin, the mucous membranes and/or the appendages.

The retinoid compound according to the invention may be selected from among all-trans retinoic acid (or tretinoin), isotretinoin or else motretinide.

The retinoid compound according to the invention is preferably selected from among naphthoic acid derivatives of formula (I), and salts and esters thereof:

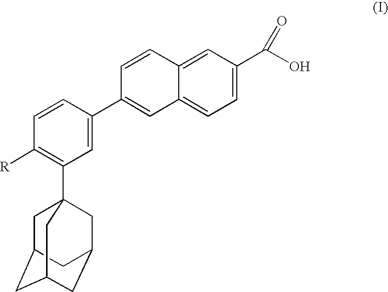

wherein R is a hydrogen atom, a hydroxyl radical, a branched or unbranched alkyl radical having from 1 to 4 carbon atoms, an alkoxy radical having from 1 to 10 carbon atoms, or a cycloaliphatic radical which is substituted or unsubstituted.

The expression “linear or branched alkyl radical having from 1 to 4 carbon atoms” means, preferably, methyl, ethyl, propyl and butyl radicals.

The expression “alkoxy radical having from 1 to 10 carbon atoms” means, preferably, methoxy, ethoxy, propoxy, butoxy, hexyloxy and decyloxy radicals.

The term “cycloaliphatic radical” means, preferably, monocyclic or polycyclic radicals, such as the 1-methylcyclohexyl radical or the 1-adamantyl radical.

The term “salts of the naphthoic acid derivatives” means salts formed with a pharmaceutically acceptable base, in particular, an inorganic base such as sodium hydroxide, potassium hydroxide or aqueous ammonia, or an organic base such as lysine, arginine or N-methylglucamine, but also the salts formed with fatty amines such as dioctylamine, aminomethyl propanol and stearylamine.

The term “esters of the naphthoic acid derivatives” means esters formed with pharmaceutically acceptable alcohols.

Preferably, among the naphthoic acid derivatives that may comprise the compositions according to the invention, 6-[3-(1-adamantyl)-4-methoxyphenyl]-2-naphthoic acid (adapalene), 6-[3-(1-adamantyl)-4-hydroxyphenyl]-2-naphthoic acid, 6-[3-(1-adamantyl)-4-decyloxyphenyl]-2-naphthoic acid or 6-[3-(1-adamantyl)-4-hexyloxyphenyl]-2-naphthoic acid will be selected.

Even more preferably, the retinoid compound that can be administered according to the invention is selected from among adapalene (6-[3-(1-adamantyl)-4-methoxyphenyl]-2-naphthoic acid), salts thereof and esters thereof.

The term “adapalene salts” means, in particular, the salts formed with a pharmaceutically acceptable base, in particular, inorganic bases such as sodium hydroxide, potassium hydroxide or aqueous ammonia, or organic bases such as lysine, arginine or N-methylglucamine.

The term “adapalene salts” also means the salts formed with fatty amines such as dioctylamine, aminomethyl propanol and stearylamine.

Preferably, the retinoid compound according to the invention is adapalene.

Advantageously, the compositions according to the invention do not comprise any depigmenting agent distinct from the retinoid compound, in particular, adapalene.

According to a specific embodiment of the invention, the adapalene is in dispersed form in the composition.

In the compositions according to the invention, the concentration of retinoid compound ranges from 0.0001% to 10%, in particular, from 0.01% to 5%, preferably from 0.01% to 1%, more preferentially from 0.01% to 0.5%, and preferentially from 0.1% to 0.3% by weight of the total weight of the composition.

Even more preferentially, the concentration of retinoid compound is equal to 0.1%. Alternatively, the concentration of retinoid compound is preferably equal to 0.3%.

According to the invention, the term “anti-irritant” means an active agent that modulates the manifestations of sensitive skin, i.e., the manifestations of skin irritation, such as stinging, tight skin, burning sensation and redness.

The expression “sensitive skin” covers both irritable and/or reactive skin and intolerant skin.

Irritable and/or reactive skin is skin which reacts through pruritis, i.e., through itching or through stinging, to various factors such as the environment, emotions, foods, wind, friction, shaving, soap, surfactants, hard water with a high calcium content, temperature variations or wool. In general, these signs are associated with dry skin with or without dry patches, or with skin that exhibits erythema.

Intolerant skin is skin which reacts through sensations of burning, tightening, stinging and/or redness, to various factors such as the environment, emotions, foods and certain cosmetic products. In general, these signs are associated with hyperseborrheic or acneic skin with or without dry patches and associated with erythema.

The use of these specific anti-irritants makes it possible to reduce the irritation caused by the active ingredients, in particular, the retinoids.

The anti-irritants that can be formulated according to the present invention are selected from among 18β-glycyrrhetinic acid (enoxolone), its salts and its derivatives.

The term “18β-glycyrrhetinic acid salts” means, in particular, the potassium salt of 18β-glycyrrhetinic acid, the sodium salt of 18β-glycyrrhetinic acid, the zinc salt of 18β-glycyrrhetinic acid, the monoammonium salt of 18β-glycyrrhetinic acid (ammonium glycyrrhetinate), the disodium succinate salt of 18β-glycyrrhetinic acid, or else the dipotassium salt of 18β-glycyrrhetinic acid.

The expression “derivatives of the 18β-glycyrrhetinic acid salts” means, in particular, the monoester of 18β-glycyrrhetinic acid.

Preferably, the anti-irritant is the potassium salt of 18β-glycyrrhetinic acid.

The anti-irritant according to the invention may be of natural or synthetic origin.

The term “natural origin” means an anti-irritant in the pure state or in solution irrespective of its concentration in said solution, obtained, by various methods, from a natural element.

The term “synthetic origin” means an anti-irritant in the pure state or in solution, irrespective of its concentration in said solution, obtained by chemical synthesis.

The concentration of anti-irritant compound according to the invention is, for its part, from 0.01% to 10%, preferentially from 0.1% to 7%.

The composition according to the invention also comprises benzoyl peroxide.

Preferably, the benzoyl peroxide according to the invention is in dispersed form.

The benzoyl peroxide that can be formulated according to the invention can equally be used in free form or else in an encapsulated form, for example, in a form adsorbed onto, or absorbed into, any porous support. It may, for example, be benzoyl peroxide encapsulated in a polymeric system consisting of porous microspheres, for instance microsponges marketed under the trademark Microsponges P009A Benzoyl Peroxide™ by Amcol.

Advantageously, the particle size of the benzoyl peroxide is such that at least 80% by number of the particles, preferably at least 90% by number of the particles, have a diameter of less than 25 μm and at least 99% by number of the particles have a diameter of less than 100 μm.

The concentration of benzoyl peroxide in the compositions according to the invention ranges from 0.0001% to 20%, preferentially from 0.025% to 10%, even more preferentially from 0.5% to 5% to more preferred 2.5% to 5%.

The composition according to the invention may also, in particular, comprise at least one propenetrating agent.

The concentration of propenetrating agents in the compositions according to the invention ranges from 0.0001% to 20%.

The propenetrating agents should generally not solubilize the active agents at the percentage used, not cause exothermic reactions harmful to the benzoyl peroxide, aid good dispersion of the active agents and have anti-foam properties.

The compositions according to the invention may also, in particular, comprise at least one pH-independent gelling agent.

The term “pH-independent gelling agent” means a gelling agent capable of conferring a sufficient viscosity on the composition so as to maintain both the retinoid, the anti-irritant and the benzoyl peroxide in suspension, even under the influence of a variation in pH due to the release of benzoic acid by the benzoyl peroxide. The gelling agent according to the invention also has good physical stability, i.e., no decrease in viscosity is observed over time at temperatures from 4 to 40° C., maintaining good chemical stability of the active agents, i.e., no degradation of the active agents is observed over time and at temperatures from 4 to 40° C.

Non-limiting examples of gelling agents and/or suspending agents and/or pH-independent agents that comprise the compositions according to the invention include microcrystalline cellulose et sodium carboxymethyl cellulose (such as this marketed as Avicel CL-611 or RC/CL by FMC Biopolymer), the “electrolyte-insensitive” carbomers marketed under the trademark Ultrez 20™, Carbopol 1382™, Pemulen TR1, Pemulen TR2 or Carbopol ETD2020™ by Noveon; polysaccharides, non-limiting examples of which include xanthan gum, such as Xantural180™ marketed by Kelco, guar gum, chitosans, carrageenans, cellulose and its derivatives such as hydroxypropylmethylcellulose, in particular, the product marketed under the trademark Methocel E4 Premium™ by Dow Chemical or hydroxyethylcellulose, in particular, the product marketed under the trademark Natrosol HHX 250™ by Aqualon, the family of magnesium aluminum silicates such as Veegum K™ marketed by Vanderbilt, the family of carrageenans in particular, those in the four following sub families: k, λ, β, ω such as Viscarin® or Gelcarins® marketed by IMCD, the family of acrylic polymers coupled to hydrophobic chains, such as the PEG-150/decyl/SMDI copolymer marketed under the trademark Aculyn 44™ (polycondensate comprising at least, as elements, a polyethylene glycol comprising 150 or 180 mol of ethylene oxide, decyl alcohol and methylenebis(4-cyclohexylisocyanate) (SMDI), at 35% by weight in a mixture of propylene glycol (39%) and water (26%)), the family of modified starches such as the modified potato starch marketed under the trademark Structure Solanace™, or else mixtures thereof, and the gelling agents of the polyacrylamide family, such as the sodium acryloyldimethyltaurate copolymer/isohexadecane/polysorbate 80 mixture marketed under the trademark Simulgel 600PHA™ by Seppic, or the polyacrylamide/isoparaffin C13-14/laureth-7 mixture such as, for example, that marketed under the trademark Sepigel 305™ by Seppic.

The preferred gelling agents are derived from the polyacrylamide family, such as Simulgel 600PHA™ or Sepigel 305™; “electrolyte-insensitive” carbomers such as Carbopol 1382™; polysaccharides such as xanthan gum; cellulose derivatives such as hydroxypropylmethylcellulose or hydroxyethylcellulose; and magnesium aluminum silicates, alone or as a mixture.

The pH-independent gelling agent as described above can be included at the preferential concentrations ranging from 0.001% to 15% to more preferentially from 0.15% to 5%.

The compositions according to the invention may also, in particular, comprise at least one wetting agent.

The wetting capacity is the tendency of a liquid to spread out over a surface.

Preferably, they are wetting agents which have an HLB (hydrophilic/lipophilic balance) of 7 to 18, or nonionic wetting agents of polyoxyethylenated and/or polyoxypropylenated copolymer type. Non-limiting examples of wetting agents include Poloxamers and more particularly the product as known as Synperonic PE/L44 (Polyethylene-polypropylene glycol; Polyoxyethylene-Polyoxypropylene Block Copolymer) and/or Synperonic PE/L62 marketed by Uniqema, glycols such as those known as propylene glycol, dipropylene glycol, lauroglycol, propylene glycol dipelargonate, ethoxydiglycol. They should be liquid so as to incorporate readily into the composition without it being necessary to heat it.

Among the wetting agents whose role it is to reduce the surface tension and to allow greater spreading of the liquid, use is preferentially made, without this list being limiting, of compounds such as those of the poloxamers and/or glycols families and more particularly Synperonic PE/L44 and/or Synperonic PE/L62 and/or compounds such as propylene glycol, dipropylene glycol, propylene glycol dipelargonate, lauroglycol, ethoxydiglycol.

By way of preferred wetting agents, exemplary are propylene glycol or synperonic PE/L44 (Poloxamer 124™).

The concentration of wetting agents in the compositions according to the invention ranges from 0.0001% to 20%, preferentially from 0.1% to 10% to more preferably from 2 and 7% in weight with regards to the total composition weight.

The compositions according to the invention may also in particular, comprise at least one emulsifier.

Preferably, the emulsifier used is different from the wetting agents.

The term “emulsifiers” means amphiphilic compounds having a hydrophobic part which has an affinity for oil and a hydrophilic part which has an affinity for water, thus creating a link from the two phases. Ionic or nonionic emulsifiers therefore stabilize emulsions (O/W) by adsorbing them into one another at the interface and forming lamellar layers of liquid crystals.

The emulsifying capacity of nonionic emulsifiers is closely linked to the polarity of the molecule. This polarity is defined by the HLB (hydrophilic/lipophilic balance).

A high HLB indicates that the hydrophilic fraction is predominant and, conversely, a low HLB indicates that the lipophilic part is predominant. For example, HLB values of greater than approximately 10 correspond to hydrophilic surfactants.

The emulsifiers may be categorized, according to their structure, under the generic terms “ionic” (anionic, cationic, amphoteric) or “nonionic”. The nonionic emulsifiers are emulsifiers which do not dissociate to ions in water and are therefore insensitive to variations in pH.

The nonionic emulsifiers are particularly suitable for the preparation of oil-in-water type emulsions. Thus, the emulsifying system comprises at least one nonionic emulsifier, with a predominant hydrophilic fraction, i.e., having a high HLB, of greater than approximately 10.

Non-limiting examples of nonionic emulsifiers having a high HLB, sorbitan esters such as the POE (20) sorbitan monooleate marketed under the trademark Tween 80™ (HLB=15), or the POE (20) sorbitan monostearate marketed under the trademark Tween 60™ (HLB=14.9), fatty alcohol ethers such as the POE (21) stearyl ether (HLB=15.5), or the ceteareth 20 marketed under the trademark Eumulgin B2™ by Cognis (HLB of 15.5) polyoxyethylene glycol esters such as glyceryl stearate and PEG 100 stearate marketed under the trademark Arlacel 165 FL® (HLB=11) by Uniqema, PEG 6 Stearate and PEG 32 stearate marketed under the trademark TEFOSE 1500 (HLB=10) by Gateffossé, sucroesters with high HLB such as PEG 20 methyl glucose sesquistearate marketed under the trademark glucamate SSE20 (HLB=15) by Amerchol and sucrose laurate marketed under the trademark Surfhope C-1216® (HLB=16) and sucrose stearate marketed under the trademark Surfhope C-1811® (HLB=11) by Gattefossé.

Preferably, said nonionic emulsifiers with a high HLB have an HLB of from 10 and 18.

Examples of nonionic emulsifiers with a low HLB (lipophilic) are sorbitan esters such as sorbitan monostearate (HLB=4.7) (marketed under the trademark Span 60™ by Uniqema company), glycerol esters such as glycerol monostearate (marketed under the trademark Cutina GMSVPH™ by Cognis company) such as glyceryl monostearate (Cutina GMS™ (HLB=3.8) from Cognis company), polyethylene glycol esters such as PEG-6 isostearate marketed with the trademark Olépal isostearic (HLB=8) by Gattefossé, sucroesters with low HLB such as methyl glucose sesquistearate marketed under the trademark Glucate SS (HLB=6) by Amerchol and sucrose dilaurate marketed under the trademark Surfhope C-1205 (HLB=5) and sucrose tristearate marketed under the trademark Surfhope C-1803 (HLB=3) by Gattefossé.

Preferably, said nonionic emulsifiers with a low HLB have an HLB of less than 10.

The nonionic emulsifiers may be used alone or as a mixture of two or more of them so as to form the emulsifying system.

Preferably, one or more “nonionic emulsifier with a high HLB”/“nonionic emulsifier with a low HLB” pairs will be used as emulsifying system; it may in particular, be a nonionic emulsifying system comprising at least one nonionic emulsifier having an HLB of greater than approximately 10 and at least one nonionic surfactant having an HLB of less than approximately 10.

The ratio of each of the two emulsifiers forming the abovementioned pair is most commonly determined by calculating the required HLB of the fatty phase used.

By way of preferred emulsifiers, exemplary are hydrophilic emulsifiers of the type glyceryl stearate & PEG-100 stearate marketed under the trademark Arlacel 165FL™ by Uniqema; the PEG 6 stearate and PEG 32 stearate marketed under the trademark Tefose 1500™ by Gattefosse, lipophilic emulsifiers of sucrose ester type, such as the glucate SS™ (methyl glucose sesquistearate) and glucamate SSE 20™ (PEG 20 methyl glucose sesquistearate) marketed by Amerchol, the polyoxyethylene (21) stearyl ether marketed under the trademark Brij721™ by Uniqema, and the ceteareth 20 marketed under the trademark Eumulgin B2PH™ by Cognis.

According to the invention, the preferred concentrations of emulsifiers are from 0.001% to 20%. More preferably, the concentration ranges from 1% to 15%, and preferably from 3% to 11% by weight, relative to the total weight of the composition.

The compositions according to the invention may also, in particular, comprise at least one chelating agent and/or at least one preservative.

Among the chelating agents, exemplary are diethylenetriaminepentaacetic acid (DTPA), ethylenediamine-di-(O-hydroxyphenylacetic) acid (EDDHA), 2-hydroxyethylenediaminetriacetic acid (HEDTA), ethylenediamine-di-(O-hydroxy-p-methylphenyl)acetic acid (EDDHMA), ethylenediaminetetraacetic acid (EDTA) and ethylenediamine-di-(5-carboxy-2-hydroxyphenyl)acetic acid (EDDCHA).

A preferred chelating agent is ethylene diamine tetraacetic acid (EDTA).

Among the preservatives, exemplary are benzoic acid and its derivatives such as benzyl alcohol, benzalkonium chloride, sodium benzoate, bronopol, chlorhexidine, chlorocresol and its derivatives, ethyl alcohol, phenethyl alcohol, phenoxyethanol, potassium sorbate, diazolidinylurea, and parabens such as propylparaben or methylparaben, taken alone or as mixtures.

By way of preferred preservative, exemplary are parabens and phenoxyethanol or benzalkonium chloride, taken alone or as a mixture.

The compositions of the invention may also, in particular, comprise any additive normally used in the cosmetics or pharmaceutical field, such as neutralizers or pH adjusters such as well known mineral or organic bases or acids, such as example triethanolamine, NaOH 10% solution, sodium succinic acid/succinate buffer, sodium citric acid/citrate buffer, humectants and/or emollients, sunscreens, antioxidants, fillers, electrolytes, dyes, customary inorganic or organic bases or acids, fragrances, essential oils, active cosmetic agents, moisturizers, vitamins, essential fatty acids, sphingolipids, self-tanning compounds such as DHA, and agents for calming and protecting the skin optionally one stabilizer of benzoyl peroxide (such as non-limited example sodium docusate, sodium C14-16 olefin sulfonate).

Of course, one skilled in the art will take care to select this or these possible additional compound(s), and/or the amount thereof, in such a way that the advantageous properties of the composition according to the invention are not, or not substantially, impaired.

The concentrations of said additives of the composition are from 0.001% to 20% by weight, relative to the total weight of the composition.

The compositions according to the present invention may be in any of the galenical forms normally employed for topical application, in particular, in the form of aqueous, aqueous-alcoholic or oily dispersions, dispersions of the lotion type, aqueous, anhydrous or lipophilic gels, emulsions of liquid consistency (in particular, compatible with a presentation form of impregnated wipe type) or semi-liquid consistency of the milk type, obtained by dispersion of a fatty phase in an aqueous phase, oil-in-water (O/W), or vice versa water-in-oil (W/O), or suspensions or emulsions of soft, semi-liquid or solid consistency, of the cream, cream-gel, foam or ointment type, or microemulsions, microcapsules, microparticles or vesicular dispersions of ionic and/or nonionic type, in the form of sprays, or else in the form of dermal devices such as patches.

The term “topical application” means application to the skin or the mucous membranes.

Preferably, the compositions according to the invention are in the form of a gel or a cream-gel of semi-liquid consistency of the milk type to solid consistency of the cream type, obtained by dispersion of a fatty phase in an aqueous phase (O/W).

Preferably, the compositions according to the invention are in the form of an emulsion, preferably a light emulsion in the form of an (O/W) emulsion.

The term “light emulsion” means an emulsion containing a low proportion of fatty phase, the aqueous phase remaining predominant.

The term “emulsion” means a liquid system comprising two fluids that are insoluble or relatively insoluble in one another, and in which one of the fluids disperses in the other in microscopic particles. Preferably, the emulsions used comprise at least one emulsifier, a polar hydrophilic, preferably aqueous, phase and a non-polar fatty phase. Preferably, they are in the form of emulsions (O/W or W/O).

To obtain this essential stabilization, an emulsifier which reduces the surface tension from the two phases is introduced. The emulsions have an important role in dermatological and cosmetic products because said emulsions correspond to the physiological needs of the skin, and make it possible to bring about uniform penetration of both water-soluble substances and oil-soluble substances.

Those skilled in the art will take care to select the excipients constituting the compositions according to the invention according to the galenical form desired, and in such a way that the advantageous properties of the composition according to the invention are respected.

The fatty phase of the composition according to the invention may comprise, for example, plant, mineral, animal or synthetic oils, silicone oils, and mixtures thereof.

Exemplary mineral oils include liquid paraffins of various viscosities, such as Primol 352®, Marcol 82® and Marcol 152® marketed by Esso.

As plant oils, exemplary are sweet almond oil, palm oil, soybean oil, sesame oil and sunflower oil.

As animal oils, exemplary are lanolin, squalene, fish oil, and mink oil, with, as a derivative, the squalane marketed under the trademark Cosbiol® by Laserson.

As synthetic oils, exemplary are an ester such as cetearyl isononanoate, for instance the product marketed under the trademark Cetiol SN PH® by Cognis France, diisopropyl adipate, for instance the product marketed under the trademark Crodamol DA® by Croda, isopropyl palmitate, for instance the product marketed under the trademark Crodamol IPP® by Croda, triglycerides such as caprylic/capric triglyceride, for instance Miglyol 812 marketed by Hüls/Univar, polymers such as hydrogenated polyisobutene and derivatives.

As volatile or non-volatile silicone oils, exemplary are dimethicones, for instance the products marketed under the trademark Dow Corning 200 Fluid® or Q7-9120 silicone fluid with a viscosity from 20 cst and 12500 cst or the product marketed under the trademark ST-Cyclomethicone-5NF by Dow corning.

Solid fatty substances such as natural or synthetic waxes, fatty acids such as stearic acid, fatty alcohols such as Speziol C18 Pharma marketed by Cognis and texture agents such as tribehenate, for example, Compritol 888 marketed by Gattefossé or hydrogenated castor oils such as Cutina HR marketed by Cognis may also be introduced. In this case, one skilled in the art will adjust the heating temperature for the preparation according to the presence or absence of these solids.

For the compositions according to the invention, synthetic and/or silicone oils, and more particularly Marcol 152® et la ST-5cyclomethicone-5NF, are preferred.

The hydrophilic phase of the emulsions according to the invention is preferably aqueous and may therefore comprise water. This water may, in particular, be a floral water such as cornflower water, or a natural mineral water or spring water, for example, selected from among Vittel water, water from the Vichy basin, Uriage water, La Roche Posay water, Avéne water or Aix-les-Bains water.

Said aqueous phase may be present at a content of from 10% to 90% by weight, relative to the total weight of the composition, preferably from 20% to 80% by weight.

The present invention also features the compositions as described above, as medicaments.

In particular, the present invention relates to the use of at least one retinoid compound, benzoyl peroxide and at least one anti-irritant compound described above, or of a composition as described above, for treatment and/or prevention of dermatological conditions linked to a keratinization disorder relating to cell differentiation and proliferation, in particular, for treating common acne, comedonic acne, papulopustular acne, papulocomedonic acne, nodulocystic acne, acne conglobata, cheloid acne of the nape of the neck, recurrent miliary acne, necrotic acne, neonatal acne, occupational acne, acne rosacea, senile acne, solar acne and acne medicamentosa.

Preferably, this invention features formulating at least one retinoid compound, benzoyl peroxide and at least one anti-irritant compound described above, or of a composition as described above, into medicaments useful for preventing and/or treating common acne.

In addition, this invention also features the cosmetic use of a composition according to the invention, for the treatment of skin with an acneic tendency, for combating the greasy appearance of the skin or the hair.

The present invention also features a method for formulating a composition as described above. Such a method comprises a step of mixing at least one retinoid compound as defined above, preferably present in a physiologically acceptable medium, with benzoyl peroxide and with at least one anti-irritant compound, said retinoid compounds and benzoyl peroxide preferably being in a dispersed form in said composition.

The introduction of the other optional excipients and additives will be carried out according to the chemical nature of the compounds and the galenical form selected.

For more clarity in the following descriptions of processes, by lipophilic compound, is meant a substance having an affinity for, tending to combine with, or capable of dissolving in lipids, fat or oils.

By hydrophilic ingredients, it is meant a substance having a strong affinity for water, tending to dissolve in, mix with, or be wetted by water.

The formulation of a composition according to the invention is carried out according to a general process as follows:

a) the retinoid compound is mixed with at least one wetting agent in water, until said retinoid compound is completely dispersed, to obtain the active phase 1;

b) the benzoyl peroxide is mixed with at least one wetting agent in water, until said benzoyl peroxide is completely dispersed, to obtain the active phase 2;

c) an aqueous phase comprising water, at least one anti-irritant, at least one hydrophilic ingredients is prepared, optionally, add the gelling agent;

d) optionally, for obtaining an emulsion, mix, if necessary heat at least one emulsifier, at least one lipophilic compound and optionally solid fatty substances until homogenization, to obtain the fatty phase;

e) Optionally, for obtaining a gel-cream, mix if necessary heat at least one oil and/or solid fatty substance until homogenization, to obtain the fatty phase;

f) the two active phases obtained respectively in a) and b) are mixed to obtain one unique active phase;

g) in case of gel or gel-cream, mix the unique active phase obtained in step f) with aqueous phase obtained in step c);

h) optionally, add the gelling agent

i) in case of emulsion, said fatty phase obtained in step d) is mixed with the aqueous active phase obtained in step c) to obtain an emulsion;

j) optionally in case of emulsion, the unique active phase obtained in step e) is mixed with emulsion obtained in step i);

k) optionally, in case of gel-cream, the unique ingredient of fatty phase or the fatty phase obtained in step e) is mixed with the phase obtained in step g) or step h);

l) if necessary, heat sensitive additives are added;

m) if necessary, a pH adjuster is introduced into the emulsion obtained in step j) or into the gel obtained in step g) or in step h) or into gel-cream obtained in step k) to obtain the desired pH;

n) if necessary, water is added to make up the remainder.

According to alternative embodiment, the composition according to the present invention is prepared as follows:

a′) steps a) and b) of the general process as described previously are merged to obtained a unique step a′) which is the mix of at least the retinoid, the benzoyl peroxide and at least one wetting agent with water until complete dispersion of ingredients to obtain a unique active phase.

Steps c), d), e), g), h), i), j), k), l), m) et n) of the previously described process remain unchanged accordingly.

More specifically, a first embodiment of the present invention is the method or the process for preparing a composition according to the invention in the form of a gel, comprising the following steps:

a) mixing at least one retinoid with water and, at least a wetting agent until complete dispersion, to obtain the active phase 1;

b) mixing the benzoyl peroxide with water and, at least a wetting agent, until complete dispersion, to obtain the active phase 2;

c) preparing an aqueous phase comprising water, at least one anti-irritant, at least one hydrophilic agent, optionally, add the gelling agent;

d) the active phases 1 and 2 respectively obtained in step a) and step b) are mixed to obtain a unique active phase;

e) the unique active phase obtained in step d) is mixed with the aqueous phase obtained in step c) and stirring until complete homogenization;

f) optionally, add the gelling agent;

g) if necessary, heat sensitive additives are added;

h) if necessary, a pH adjuster is introduced into the phase obtained in step d) or in step e) or in step f) to obtain the desired pH;

i) if necessary, water is added to make up the remainder.

More specifically, according to a particular embodiment of the invention, an alternative process of preparation of the instant composition in a form of a gel, comprises the following steps:

a′) steps a) and b) of the general process as described previously are merged to obtained a unique step a′) which is the mix of at least the retinoid, the benzoyl peroxide and at least one wetting agent with water until complete dispersion of ingredients to obtain a unique active phase.

Steps c), d), f), g), h), i) of the previously described process remain unchanged accordingly.

According to another embodiment, the method for preparing the compositions according to the invention in the form of a cream-gel, comprises successively the following steps of:

a) mixing at least one retinoid with water and, at least one wetting agent, until complete dispersion, to obtain the active phase 1;

b) mixing the benzoyl peroxide with water and, et least one a wetting agent, until complete dispersion, to obtain the active phase 2;

c) preparing an aqueous phase comprising water, at least one anti-irritant and, at least one hydrophilic agent, optionally, add the gelling agent;

d) optionally, mixing at least two lipophilic compounds to obtain the fatty phase;

e) the active phases 1 and 2 respectively obtained in step a) and step b) are mixed together to obtain a unique active phase;

f) the unique active phase obtained in step d) is mixed with the aqueous phase obtained in c)

g) optionally, add the gelling agent;

h) add the unique ingredient of fatty phase or optionally the fatty phase obtained in step d) in the gel obtained in step f) or in step g) to obtain a gel-cream;

i) if necessary, heat sensitive additives are added;

j) if necessary, a pH adjuster is introduced gel-cream obtained in step h) or in step i);

k) if necessary, water is added to make up the remainder.

More specifically, according to a particular embodiment of the invention, one aspect is an alternative process of preparation of the instant invention in a form of a gel-cream, comprising the following steps:

a′) steps a) and b) of the general process as described previously are merged to obtained a unique step a′) which is the mix of at least the retinoid, the benzoyl peroxide and at least one wetting agent with water until complete dispersion of ingredients to obtain a unique active phase.

Steps c), d), e), f), g), h), i) j), k) of the previously described process remain unchanged accordingly.

According to a third embodiment, the method for preparing the compositions according to the invention in the form of an emulsion comprises successively the following steps of:

a) mixing at least one retinoid with water and, at least one wetting agent, until complete dispersion, to obtain the active phase 1;

b) mixing the benzoyl peroxide with water and, at least one wetting agent, until complete dispersion, to obtain the active phase 2;

c) preparing an aqueous phase comprising water, at least one anti-irritant and, at least one hydrophilic agent;

d) the active phases 1 and 2 respectively obtained in step a) and step b) are mixed together to obtain a unique active phase;

e) mixing at least one emulsifier with at least one lipophilic compound to obtain the fatty phase;

f) the fatty phase obtained in step e) is mixed with the aqueous phase obtained in step c) to obtain an emulsion.

g) the unique active phase obtained in step d) is mixed with the emulsion obtained in step f)

h) optionally, add the gelling agent;

i) if necessary, heat sensitive additives are added;

j) if necessary, a pH adjuster is introduced in emulsion obtained in step h);

k) if necessary, water is added to make up the remainder.

More specifically, according to a particular embodiment of the invention, one aspect is an alternative process of preparation of the instant invention in a form of an emulsion, comprising the following steps:

a′) steps a) and b) of the general process as described previously are merged to obtained a unique step a′) which is the mix of at least the retinoid, the benzoyl peroxide and at least one wetting agent with water until complete dispersion of ingredients to obtain a unique active phase.

Steps c), d), e), f), g), h), i) j), k) of the previously described process remain unchanged accordingly.

The methods for formulating the compositions according to the invention presented above are exemplary only.

Finally, the present invention also features the non-therapeutic cosmetic treatment process for embellishing the skin or its surface appearance, in which a subject composition comprising, in a physiologically acceptable medium, a retinoid, an anti-irritant selected from among 18R-glycyrrhetinic acid (enoxolone), its salts and its derivatives and benzoyl peroxide, is topically applied to the skin and/or its appendages.

To further illustrate the present invention and the advantages thereof, the following specific examples are given, it being understood that same are intended only as illustrative and in nowise imitative. In said examples to follow, all parts and percentages are given by weight, unless otherwise indicated.

### EXAMPLES

### Example 1

### Formulation of Cream Type Comprising 0.1% Adapalene, 2.5% Benzoyl Peroxide and Enoxolone as Anti-Irritant

The formula is prepared according to above detailed process of preparation:

| Constituents | Content (% m/m) |
|---|---|
| Benzoyl Peroxide | 2.50 |
| Adapalene | 1.50 |
| Enoxolone | 1.50 |
| Synperonic PE/L44 | 0.20 |
| Sodium Docusate | 0.05 |
| Propylene glycol | 6.00 |
| EDTA | 0.10 |
| Carbopol Ultrez 20 | 0.40 |
| Glycerine | 3.00 |
| Glucamate SSE 20 | 3.50 |
| Glucate SS | 3.50 |
| Cosbiol | 6.00 |
| ST-Cyclomethicone 5 NF | 13.00 |
| Purified water | qsp 100% |
| Triethanolamine | qsp pH 5.5 ± 0.5 |

### Example 2

### Formulation of Cream Type Comprising 0.3% Adapalene, 5% Benzoyl Peroxide and Sodium Salt of 18β-Glycyrrhetinic Acid as Anti-Irritant

The formula is prepared according to above detailed process of preparation:

| Constituents | Content (% m/m) |
|---|---|
| Benzoyl Peroxide | 5.00 |
| Adapalene | 0.30 |
| Sodium Salt of 18β-Glycyrrhetinic Acid | 1.50 |
| Dipropylene glycol | 5.00 |
| Synperonic PE/L44 | 0.20 |
| Glycerine | 7.00 |
| Xantural 180 | 0.40 |
| Eumulgin B2 PH | 3.00 |
| Arlacel 165FL | 3.00 |
| Speziol C18 Pharma | 2.00 |
| Mygliol 812 N | 7.00 |
| ST-Cyclomethine 5 NF | 6.00 |
| Simulgel 600 PHA | 2.50 |
| Purified Water | qsp 100% |
| Sodium Hydroxyde | qsp pH 5.5 ± 0.5 |

### Example 3

### Formulation of Lotion Type Comprising 0.3% Adapalene, 1% Benzoyl Peroxide and the Sel Dipotassium Salt of 18β-Glycyrrhetinic Acid as Anti-Irritant

The formula is prepared according to above detailed process of preparation:

| Constituents | Content (% m/m) |
|---|---|
| Benzoyl Peroxide | 1.00 |
| Adapalene | 0.30 |
| Dipotassium salt of 18β-Glycyrrhetinic Acid | 1.50 |
| Avicel CL-611 | 1.50 |
| Dipropylene glycol | 3.00 |
| Synperonic PE/L44 | 0.20 |
| Methyl paraben | 0.15 |
| Brij 721 | 3.00 |
| Arlacel 165FL | 3.00 |
| Propyl Paraben | 0.05 |
| Perhydrosqualene | 5.00 |
| Cetiol SN PH | 5.00 |
| Simulgel 600PHA | 1.50 |
| Purified Water | qsp 100% |
| Triethanolamine | qsp pH 5.5 +/− 0.5 |

### Example 4

### Formulation of Gel Type Comprising 0.1% Adapalene, 2.5% Benzoyl Peroxide and Potassium Salt of 18β-Glycyrrhetinic Acid as Anti-Irritant

The formula is prepared according to above detailed process of preparation:

| Constituents | Content (% m/m) |
|---|---|
| Benzoyl Peroxide | 2.50 |
| Adapalene | 0.10 |
| Potassium Salt of 18β-Glycyrrhetinic Acid | 1.50 |
| Propylene Glycol | 4.00 |
| Synperonic PE/L44 | 0.20 |
| EDTA | 0.10 |
| Glycerine | 4.00 |
| Sodium Docusate | 0.05 |
| Simulgel 600PHA | 4.00 |
| Purified Water | qsp 100% |

### Example 5

### Formulation of Gel-Cream Type Comprising 0.1% Adapalene, 2.5% Benzoyl Peroxide And Potassium Salt of 18β-Glycyrrhétinic Acid as Anti-Irritant

The formula is prepared according to above detailed process of preparation:

| Constituents | Content (% m/m) |
|---|---|
| Benzoyl Peroxide | 2.50 |
| Adapalene | 0.10 |
| Potassium Salt of 18β-Glycyrrhetinic Acid | 1.50 |
| Propylene Glycol | 6.00 |
| Synperonic PE/L44 | 0.20 |
| Glycerine | 5.00 |
| ST-Cyclomethicone 5NF | 7.00 |
| Simulgel 600PHA | 4.00 |
| Purified Water | qsp 100% |

### Example 6

### Formulation of Gel Type Comprising 0.3% Adapalene, 2.5% Benzoyl Peroxide and the Monoammonium Salt of 18β-Glycyrrhetinic Acid as Anti-Irritant

The formula is prepared according to above detailed process of preparation:

| Constituents | Content (% m/m) |
|---|---|
| Adapalene | 0.30 |
| Monoammonium Salt of 18β-Glycyrrhetinic Acid | 1.50 |
| Benzoyl Peroxide | 2.50 |
| Titriplex III | 0.20 |
| Simulgel 600 | 2.00 |
| Propylene Glycol | 4.00 |
| Synperonic PE/L62 | 0.20 |
| Phenoxyethanol | 1.00 |
| Purified Water | qs 100 |
| Sodium Hydroxide 10% m/m | qspH 5.5 ± 0.5 |

### Example 7

### Formulation of Gel Type Comprising 0.3% Adapalene, 5% Benzoyl Peroxide and the Disodium Succinate Salt of 18β-Glycyrrhetinic Acid as Anti-Irritant

The formula is prepared according to above detailed process of preparation:

| Constituents | Content (% m/m) |
|---|---|
| Adapalene | 0.30 |
| Disodium Succinate salt of 18β-Glycyrrhetinic Acid | 1.50 |
| Benzoyl Peroxide | 5.00 |
| Titriplex III | 0.20 |
| Natrosol 250 HHX Pharm | 2.00 |
| Propylene Glycol | 4.00 |
| Synperonic PE/L62 | 0.20 |
| Phenoxyethanol | 1.00 |
| Purified Water | qs 100 |

### Example 8

### Formulation of Emulsion Type Comprising 0.1% Adapalene, 2.5% Benzoyl Peroxide and the Dipotassium Salt of 18β-Glycyrrhetinic Acid as Anti-Irritant

The formula is prepared according to above detailed process of preparation:

| Constituents | Content (% m/m) |
|---|---|
| Benzoyl Peroxide | 2.50 |
| Adapalene | 0.10 |
| Dipotassium Salt of 18β-Glycyrrhetinic Acid | 1.50 |
| Propylene Glycol | 2.00 |
| Synperonic PE/L62 | 0.20 |
| HEDTA | 0.10 |
| Nipagin M (optional) | 0.20 |
| Carbopol Ultrez 20 | 0.15 |
| Veegum K | 0.30 |
| Glycerol | 3.00 |
| Phenoxyethanol | 1.00 |
| Nipasol M (optional) | 0.20 |
| Glucate SS | 1.00 |
| Glucamate SSE20 | 5.00 |
| Miglyol 812 N | 9.00 |
| Q7-9120 Silicone Fluid 20 cst | 1.00 |
| Purified Water | qs 100 |
| Sodium Hydroxide 10% m/m | qs pH 5.5 ± 0.5 |

### Example 9

### Formulation of Emulsion Type Comprising 0.3% Adapalene, 2.5% Benzoyl Peroxide and the Potassium Salt of 18β-Glycyrrhetinic Acid as Anti-Irritant

The formula is prepared according to above detailed process of preparation:

| Constituents | Content (% m/m) |
|---|---|
| Benzoyl Peroxide | 2.50 |
| Adapalene | 0.30 |
| Potassium Salt of 18β-Glycyrrhetinic Acid | 1.50 |
| Propylene Glycol | 2.00 |
| Synperonic PE/L62 | 0.20 |
| HEDTA | 0.10 |
| Nipagin M (optional) | 0.20 |
| Carbopol Ultrez 20 | 0.15 |
| Veegum K | 0.30 |
| Glycerol | 3.00 |
| Phenoxyethanol | 1.00 |
| Nipasol M (optional) | 0.20 |
| Glucate SS | 1.00 |
| Glucamate SSE20 | 5.00 |
| Miglyol 812 N | 9.00 |
| Q7-9120 Silicone Fluid 20 cst | 1.00 |
| Purified Water | qs 100 |
| Sodium Hydroxide 10% m/m | qspH 5.5 ± 0.5 |

### Example 10

### Formulation of Emulsion Type Comprising 0.1% Adapalene, 2.5% Benzoyl Peroxide and 18β-Glycyrrhetinic Acid Disodium Succinate as Anti-Irritant

The formula is prepared according to above detailed process of preparation:

| Constituents | Content (% m/m) |
|---|---|
| Benzoyl Peroxide | 2.50 |
| Adapalene | 0.10 |
| 18β-Glycyrrhetinic Acid Disodium Succinate | 1.50 |
| Propylene Glycol | 4.00 |
| Synperonic PE/L44 | 0.20 |
| HEDTA | 0.10 |
| Nipagin M (optional) | 0.20 |
| Carbopol ETD2020 | 0.20 |
| Stearyl Alcohol | 1.00 |
| Phenoxyethanol | 1.00 |
| Nipasol M (optional) | 0.10 |
| Tefose 1500 | 5.00 |
| Miglyol 812 N | 7.00 |
| Purified Water | qs 100 |
| Sodium Hydroxide 10% m/m | qs pH 5.5 ± 0.5 |

### Example 11

### Formulation of Cream-Gel Type Comprising 0.3% Adapalene, 5% Benzoyl Peroxide and Enoxolone as Anti-Irritant

The formula is prepared according to above detailed process of preparation:

| Constituents | Content (% m/m) |
|---|---|
| Benzoyl Peroxide | 5.00 |
| Adapalene | 0.30 |
| Enoxolone | 2.00 |
| Propylene Glycol | 7.00 |
| Synperonic PE/L44 | 0.20 |
| HEDTA | 0.10 |
| Nipagin M (optional) | 0.20 |
| Glycerol | 5.00 |
| Simulgel 600 | 3.00 |
| Phenoxyethanol | 1.00 |
| Nipasol M (optional) | 0.10 |
| Miglyol 812 | 7.00 |
| Purified Water | qs 100 |
| Sodium Hydroxide 10% m/m | qs pH 5.5 ± 0.5 |

Each patent, patent application, publication, text and literature article/report cited or indicated herein is hereby expressly incorporated by reference in its entirety.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A topically applicable, non-irritating, emollient and rheologically stable dermatological composition comprising:
at least one retinoid compound selected from among all-trans retinoic acid, isotretinoin, motretinide, and naphthoic acid compounds of formula (I), and salts and esters thereof: 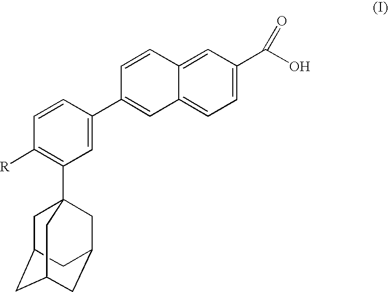
wherein R is a hydrogen atom, a hydroxyl radical, a branched or unbranched alkyl radical having from 1 to 4 carbon atoms, an alkoxy radical having from 1 to 10 carbon atoms, or a cycloaliphatic radical which is substituted or unsubstituted,
benzoyl peroxide, and
at least one anti-irritant compound selected from among 18β-glycyrrhetinic acid, its salts and its derivatives thereof,
formulated into a topically applicable, physiologically acceptable medium therefor.

2. The dermatological composition as defined by claim 1, wherein the at least one retinoid compound comprises a naphthoic acid compound of formula (I), or salt or ester thereof.

3. The dermatological composition as defined by claim 2, said compound of formula (I) comprising an alkyl radical selected from among the methyl, ethyl, propyl or butyl radical; an alkoxy radical selected from among the methoxy, ethoxy, propoxy, butoxy, hexyloxy or decyloxy radical; or a cycloaliphatic radical selected from the 1-methylcyclohexyl radical or the 1-adamantyl radical.

4. The dermatological composition as defined by claim 2, wherein said at least one retinoid compound is selected from among 6-[3-(1-adamantyl)-4-methoxyphenyl]-2-naphthoic acid (adapalene), 6-[3-(1-adamantyl)-4-hydroxyphenyl]-2-naphthoic acid, 6-[3-(1-adamantyl)-4-decyloxyphenyl]-2-naphthoic acid and 6-[3-(1-adamantyl)-4-hexyloxyphenyl]-2-naphthoic acid, and the salts and esters thereof.

5. The dermatological composition as defined by claim 4, wherein the at least one retinoid compound comprises adapalene, or salt or ester thereof.

6. The dermatological composition as defined by claim 1, wherein the concentration of retinoid compound ranges from 0.001% to 10% by weight of the total weight of the composition.

7. The dermatological composition as defined by claim 6, wherein the concentration of retinoid compound is 0.1%.

8. The dermatological composition as defined by claim 6, wherein the concentration of retinoid compound is 0.3%.

9. The dermatological composition as defined by claim 1, wherein the at least one anti-irritant compound is selected from among the potassium salt of 18β-glycyrrhetinic acid, the sodium salt of 18β-glycyrrhetinic acid, the monoammonium salt of 18β-glycyrrhetinic acid, the disodium succinate salt of 18β-glycyrrhetinic acid, the dipotassium salt of 18β-glycyrrhetinic acid and the monoester of 18β-glycyrrhetinic acid.

10. The dermatological composition as defined by claim 1, wherein the concentration of at least one anti-irritant compound ranges from 0.01% to 10%.

11. The dermatological composition as defined by claim 1, wherein the benzoyl peroxide is in dispersed form.

12. The dermatological composition as defined by claim 1, wherein the benzoyl peroxide is in encapsulated or free form.

13. The dermatological composition as defined by claim 1, comprising from 0.0001% to 20% of benzoyl peroxide.

14. The dermatological composition as defined by claim 1, formulated in the form of aqueous, aqueous-alcoholic or oily dispersions, dispersions of the lotion type, aqueous, anhydrous or lipophilic gels, emulsions of liquid or semi-liquid consistency of the milk type, obtained by dispersion of a fatty phase in an aqueous phase (O/W) or vice versa (W/O), or suspensions or emulsions of soft, semi-liquid or solid consistency of the cream, cream-gel, foam or ointment type, or microemulsions, microcapsules, microparticles or vesicular dispersions of ionic and/or nonionic type, in the form of sprays, or in the form of dermal devices and patches.

15. The dermatological composition as defined by claim 14, in the form of a gel, a cream-gel or an emulsion.

16. A regime or regimen for the treatment and/or prevention of dermatological conditions linked to a keratinization disorder relating to cell differentiation and proliferation, for treating common acne, comedonic acne, papulopustular acne, papulocomedonic acne, nodulocystic acne, acne conglobata, cheloid acne of the nape of the neck, recurrent miliary acne, necrotic acne, neonatal acne, occupational acne, acne rosacea, senile acne, solar acne and acne medicamentosa, comprising topically applying onto the skin of an individual in need of such treatment, a thus effective amount of a dermatological composition as defined by claim 1.

17. A regime or regimen as defined by claim 16, for preventing or treating common acne.

18. A regime or regimen for the treatment of skin with an acneic tendency or for combating the greasy appearance of the skin or the hair, comprising topically applying onto the skin or hair of an individual in need of such treatment, a thus effective amount of a dermatological composition as defined by claim 1.

19. A method for formulating a composition as defined by claim 1, comprising a step of mixing at least one retinoid compound with benzoyl peroxide and with at least one anti-irritant compound.

20. A non-therapeutic cosmetic regime or regimen for embellishing the skin or its surface appearance, comprising topically applying onto the skin and/or it integuments of an individual in need of such treatment, a thus effective amount of a dermatological composition as defined by claim 1.
